(19) United States
(12) Patent Application Publication
(10) Pub. No.: US 2018/0044611 A1
(43) Pub. Date: Feb. 15, 2018

(54) SULFOBETAINE-CONTAINING DETERGENTS AND CLEANING AGENTS

(71) Applicant: Henkel AG & Co. KGaA, Duesseldorf (DE)

(72) Inventors: Hendrik Hellmuth, Darmstadt (DE); Thomas Gerke, Duesseldorf (DE); Nicole Bode, Duesseldorf (DE); Michael Strotz, Koeln (DE); Michael Dreja, Neuss (DE)

(73) Assignee: Henkel AG & Co. KGaA, Duesseldorf (DE)

(21) Appl. No.: 15/792,084

(22) Filed: Oct. 24, 2017

Related U.S. Application Data
(63) Continuation of application No. PCT/EP2016/058169, filed on Apr. 14, 2016.

(30) Foreign Application Priority Data
Apr. 28, 2015 (DE) 102015207735.7

(51) Int. Cl. C11D 1/94; C07D 295/088

(52) U.S. Cl. CPC C11D 1/94; C07D 295/088; C11D 1/92

(57) ABSTRACT

The aim of the invention is to improve the cleaning performance of washing or cleaning agents with respect to soiling. This is achieved essentially by incorporating sulfobetaine-morpholine derivatives.

### FIELD OF THE INVENTION

The present invention relates to washing and cleaning agents containing certain sulfobetaine surfactants.

### BACKGROUND OF THE INVENTION

The removal of soils present on textiles is the primary objective of the textile washing operation. Likewise, the removal of soils present on hard surfaces such as dishware, glass, tile, or household work surfaces is the primary objective of the corresponding cleaning operations used. Washing or cleaning agents that are used contain for the stated purpose surfactants and generally other ingredients such as bleaching agents or enzymes, which are able to remove dirt from the textile surface or the hard surface, or to chemically modify dirt components, for example by oxidation or enzymatic degradation, so that they are easier to remove from the surface. Various efforts have focused on further improvement in the cleaning result.

It has surprisingly been found that the use of certain amphoteric morpholine derivatives contributes to improvement in the washing and cleaning result.

### BRIEF SUMMARY OF THE INVENTION

The subject matter of the invention, therefore, is a washing or cleaning agent that contains a morpholine derivative of general formula (I),

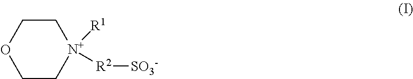

in which R¹ stands for a linear or branched alkyl functional group having 6 to 20 C atoms, in particular 12 to 16 C atoms, and R² stands for a linear or branched alkylene functional group having 2 to 20 C atoms, in particular 3 to 16 C atoms. The agent preferably contains 0.05% by weight to 20% by weight, in particular 0.1% by weight to 10% by weight, of morpholine derivative of general formula (I). In preferred compounds of general formula (I), the functional group R² is an n-propylene group or an n-butylene group in which the N atom and the —SO₃⁻ substituent are present at opposite terminal positions. In other preferred compounds of general formula (I), the functional group R¹ is a linear alkyl group.

Morpholine derivatives of general formula (I) may be produced as described in Tenside Surf. Det. 50 (2013) 1, pages 45 to 51. In particular an improvement in the removal of oil- and/or grease-containing soils is observed when these morpholine derivatives are used in washing or cleaning agents; therefore, a further subject matter of the invention is the use of the stated morpholine derivative of general formula (I) for improving the cleaning performance of washing or cleaning agents with respect to oil- and/or grease-containing soils during the washing of textiles or cleaning of hard surfaces in particular with an aqueous liquid containing builders. Within the scope of this use, it is preferred to use a washing or cleaning agent according to the invention, although it is also possible to separately add a morpholine derivative of general formula (I) and a washing or cleaning agent not containing same to a liquid, in particular water, that is provided for washing laundry or for cleaning a hard surface.

If desired, the agents according to the invention may contain even further ingredients that are customary in washing or cleaning agents, provided that they do not interact in an unacceptable manner with the morpholine derivative. Named as examples here are builders, bleaching agents, enzymes, further surfactants, solvents, and fragrances.

Within the scope of the use according to the invention, it is preferred when the concentration of morpholine derivative of general formula (I) in the aqueous liquor, as used, for example, in washing machines or dishwashers, as well as for hand laundering, hand dishwashing, or cleaning of other hard surfaces, or possibly for the cleaning of carpets or upholstery materials, is 0.005 g/L to 1 g/L, in particular 0.1 g/L to 0.5 g/L. The use according to the invention is preferably carried out at temperatures in the range of 10° C. to 95° C., in particular 20° C. to 40° C. The use according to the invention is preferably carried out at pH values in the range of pH 5 to pH 12, in particular pH 7 to pH 11.

Washing or cleaning agents, which may be present in particular as powdered solids, in re-pressed particle form, or as homogeneous solutions or suspensions, in principle may contain, in addition to the morpholine derivative to be used according to the invention, any known ingredients that are customary in these types of agents. The agents may in particular contain builder substances, further surface-active surfactants, water, water-miscible organic solvents, enzymes, sequestering agents, electrolytes, pH regulators, polymers having special effects, such as soil release polymers, dye transfer inhibitors, graying inhibitors, wrinkle-reducing and shape-retaining polymeric active ingredients, additives for improving the flow and drying behavior, corrosion inhibitors, and other auxiliaries such as optical brighteners, foam regulators, dyes, and fragrances.

The agents may contain, in addition to the morpholine derivative of general formula (I), one or more further surfactants, in particular anionic surfactants, nonionic surfactants, and the mixtures thereof being suitable, as well as cationic surfactants and/or further amphoteric surfactants. Suitable nonionic surfactants are in particular alkyl glycosides and ethoxylation and/or propoxylation products of alkyl glycosides, or linear or branched alcohols having in each case 12 to 18 C atoms in the alkyl portion and 3 to 20, preferably 4 to 10, alkyl ether groups. Also usable are corresponding ethoxylation and/or propoxylation products of N-alkylamines, vicinal diols, fatty acid esters, and fatty acid amides, which with respect to the alkyl portion correspond to the stated long-chain alcohol derivatives, and of alkyl phenols having 5 to 12 C atoms in the alkyl functional group.

Suitable anionic surfactants are in particular soaps, and anionic surfactants that contain sulfate or sulfonate groups with preferably alkali ions, in particular sodium ions, as cations. Usable soaps are preferably the alkali salts of saturated or unsaturated fatty acids having 12 to 18 C atoms. Such fatty acids may also be used in the incompletely neutralized form. Usable surfactants of the sulfate type include the salts of sulfuric acid semiesters of fatty alcohols having 12 to 18 C atoms, the so-called alkyl sulfates, and the sulfation products of the stated nonionic surfactants having a low ethoxylation number, the so-called ether sulfates. Usable surfactants of the sulfonate type include linear alkylbenzene sulfonates having 9 to 14 C atoms in the alkyl portion, alkane sulfonates having 12 to 18 C atoms, and olefin sulfonates having 12 to 18 C atoms, which result from the reaction of corresponding monoolefins with sulfur trioxide, as well as alpha-sulfofatty acid esters, which result from the sulfonation of fatty acid methyl or ethyl esters.

These types of further surfactants are contained in washing or cleaning agents in quantities of preferably 0.5% by weight to 15% by weight, in particular 1% by weight to 10% by weight, with alkoxylated alcohols, alkyl sulfates, and ether sulfates as well as mixtures of at least two of these surfactants being preferred. It is preferred when the weight ratio of morpholine derivative of general formula (I) to further surfactant, in particular alkoxylated alcohol and/or anionic surfactant, is in the range of 1:30 to 30:1, in particular 50:50 to 20:80. In likewise preferred embodiments of the invention, no nonionic surfactant is present.

Agents according to the invention for use in the washing of textiles may contain in particular one or more of the cationic, textile-softening substances of general formulas II, Ill, or IV as cationic active substances having a textile-softening effect:

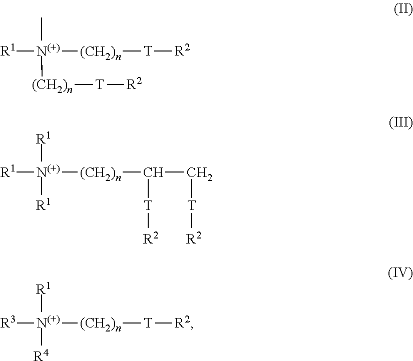

where each group R¹ is independently selected from C₁₋₆ alkyl, alkenyl, or hydroxyalkyl groups; each group R² is independently selected from C₈₋₂₈ alkyl or alkenyl groups; R³═R¹ or (CH₂)ₙ-T-R²; R⁴═R¹ or R² or (CH₂)ₙ-T-R²; T=—CH₂—, —O—CO—, or —CO—O—, and n is an integer from 0 to 5. The cationic textile-softening substances contain customary anions of a type and quantity necessary for charge balancing, and may be selected from halides, for example, as well as from anionic surfactants. In preferred embodiments, hydroxyalkyltrialkylammonium compounds, in particular C₁₂₋₁₈ alkyl(hydroxyethyl)dimethylammonium compounds, and preferably the halides thereof, in particular chlorides, are used. The agent preferably contains up to 25% by weight, in particular 0.5% by weight to 15% by weight, of cationic textile-softening substance.

A washing or cleaning agent preferably contains at least one water-soluble and/or water-insoluble, organic and/or inorganic builder. The water-soluble organic builder substances include polycarboxylic acids, in particular citric acid and sugar acids, monomeric and polymeric aminopolycarboxylic acids, in particular methyl glycine diacetic acid, nitrilotriacetic acid, and ethylenediaminetetraacetic acid, as well as polyaspartic acid, polyphosphonic acids, in particular aminotris(methylenephosphonic acid), ethylenediaminetetrakis(methylenephosphonic acid), and 1-hydroxyethane-1,1-diphosphonic acid, polymeric hydroxy compounds such as dextrin and polymeric (poly)carboxylic acids, in particular polycarboxylates that are obtainable by oxidation of polysaccharides or dextrins, and/or polymeric acrylic acids, methacrylic acids, maleic acids, and mixed polymers thereof, which may also contain small portions of polymerizable substances, not having a carboxylic acid functionality, in copolymerized form. The relative molecular mass of the homopolymers of unsaturated carboxylic acids is generally between 5,000 and 200,000, and that of the copolymers is between 2,000 and 200,000, preferably 50,000 to 120,000, in each case based on free acid. A particularly preferred acrylic acid-maleic acid copolymer has a relative molecular mass of 50,000 to 100,000. Suitable, although less preferred, compounds of this class are copolymers of acrylic acid or methacrylic acid with vinyl ethers such as vinyl methyl ethers, vinyl esters, ethylene, propylene, and styrene, in which the proportion of the acid is at least 50% by weight. Also usable as water-soluble organic builder substances are terpolymers, which contain two unsaturated acids and/or the salts thereof as monomers, and vinyl alcohol and/or an esterified vinyl alcohol or a carbohydrate as third monomer. The first acidic monomer or salt thereof is derived from a monoethylenically unsaturated C₃-C₈ carboxylic acid, preferably from a C₃-C₄ monocarboxylic acid, in particular from (meth)acrylic acid. The second acidic monomer or salt thereof may be a derivative of a C₄-C₈ dicarboxylic acid, with maleic acid being particularly preferably preferred, and/or a derivative of an allylsulfonic acid that is substituted with an alkyl or aryl functional group in the 2-position. These types of polymers generally have a relative molecular mass between 1,000 and 200,000. Further preferred copolymers are those having acrolein and acrylic acid/acrylic acid salts or vinyl acetate as monomers. The organic builder substances, in particular for producing liquid agents, may be used in the form of aqueous solutions, preferably in the form of 30 to 50% by weight aqueous solutions. All of the stated acids are generally used in the form of their water-soluble salts, in particular their alkali salts.

These types of organic builder substances may, if desired, be contained in quantities of up to 40% by weight, in particular up to 25% by weight, and preferably 1% by weight to 8% by weight. Quantities near the stated upper limit are preferably used in paste-form or liquid, in particular water-containing, agents.

Suited in particular as water-soluble inorganic builder materials are polymeric alkali phosphates, which may be present in the form of their alkaline neutral or acidic sodium or potassium salts. Examples of such include tetrasodium diphosphate, disodium dihydrogen diphosphate, pentasodium triphosphate, so-called sodium hexametaphosphate, and the corresponding potassium salts or mixtures of sodium and potassium salts. In particular crystalline or amorphous alkali aluminosilicates are used in quantities of up to 50% by weight, preferably no greater than 40% by weight, and in liquid agents, in particular in quantities of 1% by weight to 5% by weight, as water-insoluble, water-dispersible inorganic builder materials. Among these, the crystalline sodium aluminosilicates in washing agent quality, in particular zeolite A, P, and optionally X, are preferred. Quantities near the stated upper limit are preferably used in solid, particulate washing agents. Suitable aluminosilicates in particular have no particles with a grain size larger than 30 μm, and are preferably composed of at least 80% by weight of particles having a size smaller than 10 μm. Their calcium binding capacity, which may be determined according to the information from German Patent specification DE 24 12 837, for example, is generally in the range of 100 mg to 200 mg CaO per gram.

Suitable substitutes or partial substitutes for the stated aluminosilicates are crystalline alkali silicates, which may be present alone or in a mixture with amorphous silicates. The alkali silicates usable as builders preferably have a molar ratio of alkali oxide to SiO₂ of less than 0.95, in particular 1:1.1 to 1:12, and may be present in amorphous or crystalline form. Preferred alkali silicates are the sodium silicates, in particular the amorphous sodium silicates, having a Na₂O:SiO₂ molar ratio of 1:2 to 1:2.8. Preferably used as crystalline silicates, which may be present alone or in a mixture with amorphous silicates, are crystalline phyllosilicates of general formula Na₂SiₓO₂ₓ₊₁.y H₂O, in which x, the so-called modulus, is a number from 1.9 to 4 and y is a number from 0 to 20, and preferred values for x are 2, 3, or 4. Preferred crystalline phyllosilicates are those in which x in the stated general formula assumes the values 2 or 3. In particular β- as well as δ-sodium disilicates (Na₂Si₂O₅.y H₂O) are preferred. In addition, practically water-free crystalline alkali silicates produced from amorphous alkali silicates, and having the above-mentioned general formula, in which x stands for a number from 1.9 to 2.1, may be used. In another preferred embodiment, a crystalline sodium phyllosilicate having a modulus of 2 to 3 and which may be produced from sand and soda may be used. Crystalline sodium silicates having a modulus in the range of 1.9 to 3.5 are used in another preferred embodiment. In one preferred embodiment, a granular compound of alkali silicate and alkali carbonate, which is commercially available under the name Nabion® 15, for example, is used. If alkali aluminosilicate, in particular zeolite, is also present as an additional builder substance, the weight ratio of aluminosilicate to silicate, in each case based on water-free active substances, is preferably 1:10 to 10:1. In agents that contain amorphous as well as crystalline alkali silicates, the weight ratio of amorphous alkali silicate to crystalline alkali silicate is preferably 1:2 to 2:1, in particular 1:1 to 2:1.

Builder substances are preferably contained in washing or cleaning agents in quantities of up to 60% by weight, in particular 5% by weight to 40% by weight.

### DETAILED DESCRIPTION OF THE INVENTION

In one preferred embodiment, an agent according to the invention, in particular a textile washing agent according to the invention, has a water-soluble builder block. Use of the term “builder block” is understood to mean that the agents contain no further builder substances than those that are water-soluble; i.e., all builder substances contained in the agent are combined in the “block” thus characterized, in any case with exclusion of the quantities of substances that may be contained as impurities or stabilizing additives in small quantities in the remaining ingredients of the agents, as is customary in the trade. The term “water-soluble” is understood to mean that the builder block dissolves, free of residues, at the concentration that results due to the used quantity of the agent containing same, under customary conditions. At least 15% by weight and up to 55% by weight, in particular 25% by weight to 50% by weight, of water-soluble builder block is preferably contained in the agents. The builder block is preferably composed of the following components:

a) 5% by weight to 35% by weight citric acid, alkali citrate, and/or alkali carbonate, which may also be replaced at least in part by alkali hydrogen carbonate,
b) up to 10% by weight alkali silicate having a modulus in the range of 1.8 to 2.5,
c) up to 2% by weight phosphonic acid and/or alkali phosphonate,
d) up to 50% by weight alkali phosphate, and
e) up to 10% by weight polymeric polycarboxylate,
wherein the stated quantities refer to the overall agent. This also applies for all of the stated quantities below, unless expressly indicated otherwise.

In one preferred embodiment, the water-soluble builder block contains at least two of the components b), c), d), and e) in quantities greater than 0% by weight.

With regard to component a), in one preferred embodiment 15% by weight to 25% by weight alkali carbonate, which may be replaced at least in part by alkali hydrogen carbonate, and up to 5% by weight, in particular 0.5% by weight to 2.5% by weight, citric acid and/or alkali citrate are contained. In one alternative embodiment, 5% by weight to 25% by weight, in particular 5% by weight to 15% by weight, citric acid and/or alkali citrate and up to 5% by weight, in particular 1% by weight to 5% by weight, alkali carbonate, which may be replaced at least in part by alkali hydrogen carbonate, are contained as component a). If both alkali carbonate and alkali hydrogen carbonate are present, component a) contains alkali carbonate and alkali hydrogen carbonate preferably in a weight ratio of 10:1 to 1:1.

With regard to component b), in one preferred embodiment 1% by weight to 5% by weight alkali silicate having a modulus in the range of 1.8 to 2.5 is contained.

With regard to component c), in one preferred embodiment 0.05% by weight to 1% by weight phosphonic acid and/or alkali phosphonate is contained. Phosphonic acids are also understood to mean substituted alkylphosphonic acids, which may also have multiple phosphonic acid groups (so-called polyphosphonic acids). They are preferably selected from the hydroxy- and/or aminoalkylphosphonic acids and/or the alkali salts thereof, for example dimethylaminomethane diphosphonic acid, 3-aminopropane-1-hydroxy-1,1-diphosphonic acid, 1-amino-1-phenylmethane diphosphonic acid, 1-hydroxyethane-1,1-diphosphonic acid, amino-tris(methylenephosphonic acid), N,N,N′,N′-ethylenediamine-tetrakis(methylenephosphonic acid), and acylated derivatives of phosphorous acid, which may also be used in any desired mixtures.

With regard to component d), in one preferred embodiment 15% by weight to 35% by weight alkali phosphate, in particular trisodiumpolyphosphate, is contained. “Alkali phosphate” is the collective term for the alkali metal (in particular sodium and potassium) salts of the various phosphoric acids, for which a distinction may be made between metaphosphoric acids (HPO₃)ₙ and orthophosphoric acid (H₃PO₄) in addition to higher-molecular representatives. The phosphates combine several advantages: they act as alkali carriers, prevent lime deposits on machine parts and lime encrustations in fabrics, and in addition contribute to the cleaning performance. Sodium dihydrogen phosphate, NaH₂PO₄, exists as the dihydrate (density 1.91 gcm⁻³, melting point 60°) and as the monohydrate (density 2.04 gcm⁻³). Both salts are white powders that are very soluble in water, lose the water of crystallization upon heating, and at 200° C. convert to the weakly acidic diphosphate (disodium hydrogen diphosphate, Na₂H₂P₂O₇), and at higher temperature convert to sodium trimetaphosphate (Na₃P₃O₉) and Madrell's salt. NaH₂PO₄ reacts acidically, and is formed when phosphoric acid is set to a pH of 4.5 with sodium hydroxide solution and the slurry is atomized. Potassium dihydrogen phosphate (primary or monobasic potassium phosphate, potassium diphosphate (KDP)), KH₂PO₄, is a white salt having a density of 2.33 gcm⁻³ and a melting point of 253° (decomposition with formation of (KPO₃)ₓ, potassium polyphosphate), and is readily soluble in water. Disodium hydrogen phosphate (secondary sodium phosphate), Na₂HPO₄, is a colorless crystalline salt that is very soluble in water. It exists in water-free form, and with 2 mol water (density 2.066 gcm⁻³, with loss of water at 95°), with 7 mol water (density 1.68 gcm⁻³, melting point 48° with loss of 5 H₂O), and with 12 mol water (density 1.52 gcm⁻³, melting point 35° with loss of 5 H₂O), is water-free at 100°, and under fairly strong heating converts to the diphosphate Na₄P₂O₇. Disodium hydrogen phosphate is produced by neutralization of phosphoric acid with soda solution, using phenolphthalein as indicator. Dipotassium hydrogen phosphate (secondary or dibasic potassium phosphate), K₂HPO₄, is an amorphous white salt that is readily soluble in water. Trisodium phosphate, and tertiary sodium phosphate, Na₃PO₄, are colorless crystals, which as the dodecahydrate have a density of 1.62 gcm⁻³ and a melting point of 73-76° C. (decomposition), as the decahydrate (corresponding to 19-20% P₂O₅) have a melting point of 100° C., and in water-free form (corresponding to 39-40% P₂O₅) have a density of 2.536 gcm⁻³. Trisodium phosphate is readily soluble in water under an alkaline reaction, and is produced by evaporating a solution of exactly 1 mol disodium phosphate and 1 mol NaOH. Tripotassium phosphate (tertiary or tribasic potassium phosphate), K₃PO₄, is a white, deliquescent, granular powder having a density of 2.56 gcm⁻³ and a melting point of 1340°, and is readily soluble in water with an alkaline reaction. It is produced, for example, by heating Thomas slag with coal and potassium sulfate. Despite the higher cost, the more readily soluble and therefore highly effective potassium phosphate is often preferred over corresponding sodium compounds. Tetrasodium diphosphate (sodium pyrophosphate), Na₄P₂O₇, exists in water-free form (density 2.534 gcm⁻³, melting point 988°, also given as 880°) and as the decahydrate (density 1.815-1.836 gcm⁻³, melting point 94° with loss of water). The substances are colorless crystals that are soluble in water with an alkaline reaction. Na₄P₂O₇ is produced by heating disodium phosphate to >200° or by reacting phosphoric acid with soda in a stoichiometric ratio and dehydrating the solution by atomization. The decahydrate complexes heavy metal salts and hardeners, and thus reduces the hardness of the water. Potassium diphosphate (potassium pyrophosphate), K₄P₂O₇, exists in the form of the trihydrate and is a colorless, hygroscopic, water-soluble powder having a density of 2.33 gcm⁻³, the pH of the 1% solution being 10.4 at 25°. Condensation of the NaH₂PO₄ or the KH₂PO₄ results in higher-molecular sodium phosphates or potassium phosphates, respectively, for which a distinction may be made between cyclic representatives, the sodium or potassium metaphosphates, and chain-based types, the sodium or potassium polyphosphates. Numerous names are used in particular for the latter: fused or calcined phosphates, Graham's salt, Kurrol's salt, and Madrell's salt. All higher sodium and potassium phosphates are collectively referred to as condensed phosphates. The commercially important pentasodium triphosphate, Na₅P₃O₁₀ (sodium tripolyphosphate), is a white salt that is either water-free, or nonhygroscopic and water-soluble with crystallization with 6 H₂O, and has the general formula NaO—[P(O)(ONa)—O]ₙ—Na, where n=3. In 100 g water, approximately 17 g of the salt, free of water of crystallization, dissolves at room temperature; approximately 20 g dissolves at 60°; and approximately 32 g dissolves at 100°. After the solution is heated at 100° for two hours, approximately 8% orthophosphate and 15% diphosphate result due to hydrolysis. In the production of pentasodium triphosphate, phosphoric acid is reacted with soda solution or sodium hydroxide solution in a stoichiometric ratio, and the solution is dehydrated by atomization. Similarly as for Graham's salt and sodium diphosphate, pentasodium triphosphate dissolves many insoluble metal compounds (also lime soaps, etc.). Pentapotassium triphosphate, K₅P₃O₁₀ (potassium tripolyphosphate), is marketed, for example, in the form of a 50% by weight solution (>23% P₂O₅, 25% K₂O). Sodium potassium tripolyphosphates also exist which are likewise usable within the scope of the present invention. These compounds are produced, for example, by hydrolyzing sodium trimetaphosphate with KOH:

(NaPO₃)₃+2KOH→Na₃K₂P₃O₁₀+H₂O

These compounds are usable in exactly the same way as sodium tripolyphosphate, potassium tripolyphosphate, or mixtures of the two; mixtures of sodium tripolyphosphate and sodium potassium tripolyphosphate, or mixtures of potassium tripolyphosphate and sodium potassium tripolyphosphate, or mixtures of sodium tripolyphosphate and potassium tripolyphosphate and sodium potassium tripolyphosphate are usable.

With regard to component e), in one preferred embodiment of the agents, 1.5% by weight to 5% by weight polymeric polycarboxylate, in particular selected from the polymerization or copolymerization products of acrylic acid, methacrylic acid, and/or maleic acid, is contained. Particularly preferred among these are the homopolymers of acrylic acid, and among these, in turn those having an average molar mass in the range of 5,000 D to 15,000 D (PA standard) are particularly preferred.

As enzymes that are usable in the agents, those from the class of the proteases, lipases, cutinases, amylases, pullulanases, mannanases, cellulases, hemicellulases, xylanases, and peroxidases and the mixtures thereof are suitable, for example proteases such as serin proteases, in particular subtilases, particularly preferably subtilisins, which may be a wild-type enzyme or a subtilisin variant, wherein the wild-type enzyme or the starting enzyme of the variant is preferably selected from the alkaline protease from Bacillus amyloliquefaciens (BPN′), the alkaline protease from Bacillus licheniformis (subtilisin Carlsberg), the alkaline protease PB92, subtilisin 147 and/or 309 (Savinase®), the alkaline protease from Bacillus lentus, preferably from Bacillus lentus (DSM 5483), the alkaline protease from Bacillus alcalophilus (DSM 11233), the alkaline protease from Bacillus gibsonii (DSM 14391) or an alkaline protease that is at least 70% identical thereto, the alkaline protease from Bacillus sp. (DSM 14390) or an alkaline protease that is at least 98.5% identical thereto, and the alkaline protease from Bacillus sp. (DSM 14392) or an alkaline protease that is at least 98.1% identical thereto, amylases such as Termamyl®, Amylase-LT®, Maxamyl®, Duramyl®, and/or Purafect® OxAm, lipases such as Lipolase®, Lipomax®, Lumafast®, Lipozym®, and/or Lipex®, and cellulases such as Celluzyme® and/or Carezyme®. Enzymatic active ingredients obtained from fungi or bacteria, such as Bacillus subtilis, Bacillus licheniformis, Streptomyces griseus, Humicola lanuginosa, Humicola insolens, Pseudomonas pseudoalcaligenes, or Pseudomonas cepacia, are particularly suited. The optionally used enzymes may be adsorbed onto carrier substances and/or embedded in shell substances to protect them from premature inactivation. The enzymes are contained in washing or cleaning agents in quantities of preferably up to 10% by weight, in particular 0.2% by weight to 2% by weight.

In one preferred embodiment, the agent contains 5% by weight to 50% by weight, in particular 8 to 30% by weight, of anionic and/or nonionic surfactant, up to 60% by weight, in particular 5 to 40% by weight, of builder substance, and 0.2% by weight to 2% by weight of enzyme selected from the lipases, cutinases, amylases, pullulanases, mannanases, cellulases, oxidases, and peroxidases, and the mixtures thereof.

The organic solvents that are usable in the washing or cleaning agents, in particular when the latter are present in liquid or pasty form, include alcohols having 1 to 4 C atoms, in particular methanol, ethanol, isopropanol, and tert-butanol, diols having 2 to 4 C atoms, in particular ethylene glycol and propylene glycol, and the mixtures thereof and the ethers that are derivable from the stated compound classes. These types of water-miscible solvents are present in the agents preferably in quantities no greater than 30% by weight, in particular 6% by weight to 20% by weight.

Examples of polymers found in nature that may be used in aqueous liquid agents as thickening agents include agar-agar, carrageenan, tragacanth, gum arabic, alginates, pectins, polyoses, guar flour, carob gum flour, starch, dextrins, gelatin, and casein, cellulose derivatives such as carboxymethylcellulose, hydroxyethylcellulose, and hydroxypropylcellulose, and polymeric polysaccharide thickening agents such as xanthan; also suitable as thickeners are fully synthetic polymers such as polyacrylic and polymethacrylic compounds, vinyl polymers, polycarboxylic acids, polyethers, polyimines, polyamides, and polyurethane.

For setting a desired pH that results from mixing the remaining components, and not by itself, the agents may contain acids that are compatible with the system and the environment, in particular citric acid, acetic acid, tartaric acid, malic acid, lactic acid, glycolic acid, succinic acid, glutaric acid, and/or adipic acid, as well as mineral acids, in particular sulfuric acid, or bases, in particular ammonium hydroxide or alkali hydroxide. These types of pH regulators are contained in the agents preferably in a quantity not greater than 20% by weight, in particular 1.2% by weight to 17% by weight.

Examples of polymers with soil-removing capability, which are often referred to as soil release active ingredients, or as soil repellents due to their ability to provide the treated surface, for example the fiber, with soil-repelling properties, include nonionic or cationic cellulose derivatives. The polymers with in particular polyester-active soil-removing capability include copolyesters of dicarboxylic acids, for example adipic acid, phthalic acid, or terephthalic acid, diols, for example ethylene glycol or propylene glycol, and polydiols, for example polyethylene glycol or polypropylene glycol. Preferably used polyesters with soil-removing capability include those compounds that are formally available by esterification of two monomer portions, the first monomer being a dicarboxylic acid, HOOC-Ph-COOH, and the second monomer being a diol, HO—(CHR¹¹—)ₐOH, which may also be present as a polymeric diol, HO—(O—(CHR¹¹—)ₐ)_{b}OH. In the formula, Ph means an o-, m-, or p-phenylene functional group that may bear 1 to 4 substituents selected from alkyl functional groups having 1 to 22 C atoms, sulfonic acid groups, carboxyl groups, and the mixtures thereof, R¹¹ means hydrogen, an alkyl functional group having 1 to 22 C atoms, and the mixtures thereof, a means a number from 2 to 6, and b means a number from 1 to 300. Monomer diol units —O—(CHR¹¹—)ₐO— as well as polymer diol units —O—(O—(CHR¹¹—)ₐ)_{b}O— are preferably present in the polyesters that are obtainable therefrom. The molar ratio of monomer diol units to polymer diol units is preferably 100:1 to 1:100, in particular 10:1 to 1:10. In the polymer diol units, the degree of polymerization b is preferably in the range of 4 to 200, in particular 12 to 140. The molecular weight or the average molecular weight or the maximum of the molecular weight distribution of preferred polyesters with soil-removing capability is in the range of 250 to 100,000, in particular 500 to 50,000. The acid on which the functional group Ph is based is preferably selected from terephthalic acid, isophthalic acid, phthalic acid, trimellitic acid, mellitic acid, the isomers of sulfophthalic acid, sulfoisophthalic acid, and sulfoterephthalic acid, and the mixtures thereof. If their acid groups are not part of the ester bonds in the polymer, they are preferably present in salt form, in particular as an alkali or ammonium salt. Among these, the sodium and potassium salts are particularly preferred. If desired, instead of the monomer HOOC-Ph-COOH, small quantities, in particular no greater than 10 mol-% based on the proportion of Ph having the meaning given above, of other acids that have at least two carboxyl groups may be used in the polyester with soil-removing capability. Examples of these acids include alkylene and alkenylene dicarboxylic acids such as malonic acid, succinic acid, fumaric acid, maleic acid, glutaric acid, adipic acid, pimelinic acid, suberic acid, azelaic acid, and sebacic acid. The preferred diols HO—(CHR¹¹—)ₐOH include those in which R¹¹ is hydrogen and a is a number from 2 to 6, and those in which a has the value 2 and R¹¹ is selected from hydrogen and the alkyl functional groups having 1 to 10, in particular 1 to 3, C atoms. Among the latter-mentioned diols, those of formula HO—CH₂—CHR¹¹—OH in which R¹¹ has the meaning given above are particularly preferred. Examples of diol components are ethylene glycol, 1,2-propylene glycol, 1,3-propylene glycol, 1,4-butanediol, 1,5-pentanediol, 1,6-hexanediol, 1,8-octanediol, 1,2-decanediol, 1,2-dodecanediol, and neopentyl glycol. Among the polymeric diols, polyethylene glycol, having an average molar mass in the range of 1,000 to 6,000, is particularly preferred. If desired, these polyesters may also be end group-terminated, wherein alkyl groups having 1 to 22 C atoms and esters of monocarboxylic acids are suitable as end groups. The end groups bound via ester bonds may be based on alkyl-, alkenyl-, and arylmonocarboxylic acids having 5 to 32 C atoms, in particular 5 to 18 C atoms. These include valeric acid, caproic acid, enanthic acid, caprylic acid, pelargonic acid, capric acid, undecanoic acid, undecenoic acid, lauric acid, lauroleic acid, tridecanoic acid, myristic acid, myristoleic acid, pentadecanoic acid, palmitic acid, stearic acid, petroselinic acid, petroseleaidic acid, oleic acid, linoleic acid, linolelaidic acid, linolenic acid, eleostearic acid, arachidic acid, gadoleinic acid, arachidonic acid, behenic acid, erucic acid, brassidic acid, clupanodonic acid, lignoceric acid, cerotic acid, melissic acid, and benzoic acid, which may bear 1 to 5 substituents having a total of up to 25 C atoms, in particular 1 to 12 C atoms, for example tert-butylbenzoic acid. The end groups may also be based on hydroxymonocarboxylic acids having 5 to 22 C atoms, including, for example, hydroxyvaleric acid, hydroxycaproic acid, ricinoleic acid, the hydrogenation product thereof, hydroxystearic acid, and o-, m-, and p-hydroxybenzoic acid. The hydroxymonocarboxylic acids may be joined together via their hydroxyl group and their carboxyl group, and may thus be present multiple times in an end group. The number of hydroxymonocarboxylic acid units per end group, i.e., their degree of oligomerization, is preferably in the range of 1 to 50, in particular 1 to 10. In one preferred embodiment of the invention, polymers of ethylene terephthalate and polyethylene oxide terephthalate, in which the polyethylene glycol units have molecular weights of 750 to 5,000 and the molar ratio of ethylene terephthalate to polyethylene oxide terephthalate is 50:50 to 90:10, are used, alone or in combination with cellulose derivatives.

Dye transfer inhibitors that are suitable for use in agents for the washing of textiles include in particular polyvinylpyrrolidones, polyvinylimidazoles, polymeric N-oxides such as poly(vinylpyridine-N-oxide), and copolymers of vinylpyrrolidone with vinyl imidazole and optionally further monomers.

The agents may contain anti-crease agents, since textile fabrics, in particular made of rayon, wool, cotton, and the mixtures thereof tend to crease due to the sensitivity of the individual fibers to bending, folding, pressing, and squeezing transversely with respect to the fiber direction. Anti-crease agents include, for example, synthetic products based on fatty acids, fatty acid esters, fatty acid amides, fatty acid alkylol esters, fatty acid alkylol amides, or fatty alcohols, which are usually reacted with ethylene oxide, or products based on lecithin or modified phosphoric acid esters.

The function of graying inhibitors is to keep dirt, which is removed from the hard surface and in particular from the textile fiber, suspended in the liquor. Water-soluble colloids, usually organic, are suited for this purpose, for example starch, glue, gelatin, salts of ethercarboxylic acids or ethersulfonic acids of the starch or the cellulose, or salts of acidic sulfuric acid esters of cellulose or starch. Polyamides containing water-soluble acidic groups are also suited for this purpose. In addition, starch derivatives other than those stated above may be used, for example aldehyde starches. Preferred are cellulose ethers such as carboxymethylcellulose (Na salt), methylcellulose, hydroxyalkylcellulose, and mixed ethers such as methylhydroxyethylcellulose, methylhydroxypropylcellulose, methylcarboxymethylcellulose, and the mixtures thereof, for example in quantities of 0.1 to 5% by weight, based on the agents.

The agents may contain optical brighteners, among these in particular derivatives of diaminostilbene disulfonic acid or the alkali metal salts thereof. For example, salts of 4,4′-bis(2-anilino-4-morpholino-1,3,5-triazinyl-6-amino)stilbene-2,2′-disulfonic acid or compounds having a similar structure are suitable, which instead of the morpholino group bear a diethanolamino group, a methylamino group, an anilino group, or a 2-methoxyethylamino group. In addition, brighteners of the substituted diphenyl styryl type may be present, for example the alkali salts of 4,4′-bis(2-sulfostyryl) diphenyl, 4,4′-bis(4-chloro-3-sulfostyryl) diphenyl, or 4-(4-chlorostyryl)-4′-(2-sulfostyryl) diphenyl. Mixtures of the above-mentioned optical brighteners may also be used.

In particular for use in machine washing, it may be advantageous to add customary foam inhibitors to the agents. For example, soaps of natural or synthetic origin having a high proportion of C₁₈-C₂₄ fatty acids are suitable as foam inhibitors. Examples of suitable nonsurfactant-like foam inhibitors are organopolysiloxanes and the mixtures thereof with microfine, optionally silanized silicic acid, as well as paraffins, waxes, microcrystalline waxes, and the mixtures thereof with silanized silicic acid or bis-fatty acid alkylene diamides. Mixtures of various foam inhibitors are also advantageously used, for example those from silicones, paraffins, or waxes. The foam inhibitors, in particular silicone- and/or paraffin-containing foam inhibitors, are preferably bound to a granular carrier substance that is soluble or dispersible in water. Mixtures of paraffins and bis-stearyl ethylene diamide are particularly preferred.

In particular organic peracids or peracidic salts of organic acids, such as phthalimidopercaproic acid, perbenzoic acid, or salts of diperdodecanedioic acid, hydrogen peroxide, and inorganic salts that release hydrogen peroxide under the washing conditions, such as perborate, percarbonate, and/or persilicate, are suitable as peroxygen compounds optionally contained in the agents, in particular the agents in solid form. Hydrogen peroxide may also be generated using an enzymatic system, i.e., an oxidase and its substrate. If solid peroxygen compounds are to be employed, these may be used in the form of powders or granules, which may also be encased in a manner known in principle. Alkali percarbonate, alkali perborate monohydrate, alkali perborate tetrahydrate, or hydrogen peroxide in the form of aqueous solutions containing 3% by weight to 10% by weight hydrogen peroxide are particularly preferably used. Peroxygen compounds are preferably present in quantities of up to 50% by weight, in particular 5% by weight to 30% by weight, in washing or cleaning agents.

In addition, customary bleach activators, which form peroxocarboxylic acids or peroxoimidic acids under perhydrolysis conditions, and/or customary transition metal complexes that activate the bleach, may be used. The component of the bleach activators that is optionally present, in particular in quantities of 0.5% by weight to 6% by weight, includes the customarily used N- or O-acyl compounds, for example multiply acylated alkylene diamines, in particular tetraacetyl ethylene diamine, acylated glycolurils, in particular tetraacetyl glycoluril, N-acylated hydantoins, hydrazides, triazoles, urazoles, diketopiperazines, sulfuryl amides, and cyanurate, in addition to carboxylic acid anhydrides, in particular phthalic acid anhydride, carboxylic acid esters, in particular sodium isononanoyl phenol sulfonate, and acylated sugar derivatives, in particular pentaacetyl glucose, as well as cationic nitrile derivatives such as trimethylammonium acetonitrile salts. To avoid interaction with the per-compounds during storage, the bleach activators may have been coated with shell substances or granulated in a known manner, wherein tetraacetyl ethylene diamine that is granulated using carboxymethylcellulose, and having average grain sizes of 0.01 mm to 0.8 mm, granulated 1,5-diacetyl-2,4-dioxohexahydro-1,3,5-triazine, and/or trialkylammonium acetonitrile provided in particle form, is particularly preferred. These types of bleach activators are preferably contained in washing or cleaning agents in quantities of up to 8% by weight, in particular 2% by weight to 6% by weight, in each case based on the overall agent.

In one preferred embodiment, an agent according to the invention is liquid, and in addition to the morpholine derivative contains up to 30% by weight, in particular 2% by weight to 15% by weight, of surfactant, up to 3% by weight, in particular 0.05% by weight to 1% by weight, of thickener, up to 10% by weight, in particular 0.1% by weight to 3% by weight, of fragrance, up to 0.1% by weight of dye, up to 5% by weight, in particular 0.5% by weight to 4.5% by weight, of one or more enzymes, and water to make 100% by weight.

The production of solid agents poses no difficulties, and in principle may take place in a known manner, for example by spray drying or granulation. For producing the agents with increased bulk weight, in particular in the range of 650 g/L to 950 g/L, a method having an extrusion step is preferred. Washing or cleaning agents in the form of aqueous solutions or other solutions containing customary solvents are produced in a particularly advantageous manner by simple mixing of the ingredients, which may be added as the substance or as a solution into an automatic mixer. In one preferred embodiment of agents for washing or cleaning in particular by machine, the agents are in the form of tablets.

### EXAMPLES

### Example 1

TABLE 1
Washing agent compositions (values in % by weight)

| | A | B | C | D | E | F | G | H |
|---|---|---|---|---|---|---|---|---|
| C₉₋₁₃ alkylbenzene sulfonate, Na salt | 9 | 10 | 6 | 7 | 5 | 15 | 15 | 9 |
| C₁₂₋₁₈ fatty alcohol with 7 EO | 8 | 9 | 6 | 7 | 5 | 6 | 11 | 10 |
| C₁₂₋₁₄ fatty alcohol sulfate with 2EO | — | — | 8 | 7 | 10 | 2 | 2 | 5 |
| C₁₂₋₁₈ fatty acid, Na salt | 4 | 3 | 3 | 3 | 4 | 2 | 4 | 7 |
| Morpholine derivative essential to the | 3 | 3 | 3 | 3 | 3 | 3 | 3 | 3 |
| invention | | | | | | | | |
| Citric acid | 2 | 3 | 3 | 2 | 2 | 2 | 2 | 3 |
| Sodium hydroxide, 50% | 3 | 3 | 2 | 3 | 3 | 3 | 3 | 4 |
| Boric acid | 1 | 1 | 1 | 1 | 1 | 1 | 1 | 1 |
| Enzymes (amylase, protease, cellulase) | + | + | + | + | + | + | + | + |
| Fragrance | 1 | 0.5 | 0.5 | 1 | 1 | 1 | 1 | 1 |
| Glycerin | 3 | 2 | 2 | 2 | 2 | — | — | 2 |
| Propanediol | — | — | — | — | — | 5 | 5 | — |
| Ethanol | 1.5 | 1.5 | 1.5 | 1.5 | 1.5 | 1.5 | 1.5 | 5 |
| PVA/maleic acid copolymer | 0.1 | — | 0.1 | — | — | — | — | — |
| Optical brightener | — | 0.1 | — | 0.1 | 0.2 | 0.2 | 0.2 | 0.2 |
| Alkylaminophosphonic acid | 0.5 | 0.5 | 0.5 | 0.5 | 0.5 | 0.5 | 0.5 | 0.5 |

| | |
|---|---|
| Water | to make 100 |

### Example 2: Wash Test

The following water-containing liquid washing agents were used, which otherwise had the identical composition: liquid washing agent V1, containing 6% by weight of Na—C₁₂₋₁₄-alkyl-7 EO-sulfate, and liquid washing agents E1 to E3, instead using 3% by weight of the morpholine derivative given in Table 2 below, and 3% by weight of Na—C₁₂₋₁₄-alkyl-7 EO sulfate.

Standardized soils on cotton were washed with one of these agents at 40° C., rinsed, and dried, followed by spectrophotometric measurement of their reflectance values (Minolta® CR400-1) (wash time 1 h, washing agent metered quantity 4.12 g/L, 5 determinations of the brightness value Y). The values shown in Table 2 represent the ΔY values of the reflectance measurement when agents E1 to E3 were used, compared to washing agent V1; higher values signify better washability. The following greasy soils were used:

### Beef fat (I)

Pork lard, colored (II)
Palm fat, colored (Ill)

### Pigment/sebum (IV)

TABLE 2

| | soil | | | |
|---|---|---|---|---|
| Agent with | I | II | Ill | IV |
| E1 3-(N-dodecylmorpholino)-1-propane sulfate | 2.6 | 1.6 | 1.4 | 6.4 |
| E2 3-(N-tetradecylmorpholino)-1-propane sulfate | 6.6 | 4.6 | 2.7 | 8.8 |
| E3 3-(N-hexadecylmorpholino)-1-propane sulfate | 6.3 | 3.5 | 3.6 | 5.8 |

What is claimed is:

1. A washing or cleaning agent comprising a morpholine derivative of general formula (I), 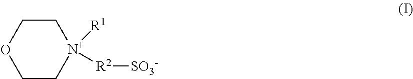
in which R¹ stands for a linear or branched alkyl functional group having 6 to 20 C atoms, and R² stands for a linear or branched alkylene functional group having 2 to 20 C atoms.

2. The agent according to claim 1, comprising 0.05% by weight to 20% by weight of morpholine derivative of general formula (I).

3. The agent according to claim 1, comprising further surfactants in quantities of 0.5% by weight to 15% by weight.

4. The agent according to claim 3, wherein the weight ratio of morpholine derivative of general formula (I) to further surfactants is from 1:30 to 30:1.

5. The agent according to claim 1, wherein it is liquid, and in addition to the morpholine derivative, comprises up to 30% by weight of surfactant, up to 3% by weight of thickener, up to 10% by weight of fragrance, up to 0.1% by weight of dye, up to 5% by weight of one or more enzymes, and water to make 100% by weight.

6. The agent according to claim 1, wherein, in the morpholine derivative of general formula (I), R¹ stands for an alkyl functional group having 12 to 16 C atoms.

7. The agent according to claim 1, wherein, in the morpholine derivative of general formula (I), R² stands for an alkylene functional group having 3 to 16 C atoms.

8. The agent according to claim 2, characterized in that it comprises 0.1% by weight to 10% by weight of morpholine derivative of general formula (I).

9. The agent according to claim 3, further comprising alkoxylated alcohols, alkyl sulfates, and ether sulfates and mixtures thereof, in quantities of 1% by weight to 10% by weight.

10. The agent according to claim 4, wherein the weight ratio of morpholine derivative of general formula (I) to alkoxylated alcohol and/or anionic surfactant is in the range of 50:50 to 20:80.

11. The agent according to claim 5, wherein it is liquid, and in addition to the morpholine derivative comprises 2% by weight to 15% by weight of surfactant, 0.05% by weight to 1% by weight of thickener, 0.1% by weight to 3% by weight of fragrance, up to 0.1% by weight of dye, 0.5% by weight to 4.5% by weight of one or more enzymes, and water t
